(19) United States
(12) Patent Application Publication
(10) Pub. No.: US 2019/0223852 A1
(43) Pub. Date: Jul. 25, 2019

(54) SURGICAL DEVICES

(71) Applicant: GYRUS MEDICAL LIMITED, Cardiff (GB)

(72) Inventor: Tony ATWELL, Newport (GB)

(73) Assignee: GYRUS MEDICAL LIMITED, Cardiff (GB)

(21) Appl. No.: 16/251,626

(22) Filed: Jan. 18, 2019

(30) Foreign Application Priority Data
Jan. 22, 2018 (GB) 1800976.1

(51) Int. Cl. A61B 17/00; A61B 17/221; A61B 17/34

(52) U.S. Cl. CPC A61B 17/00234; A61B 17/221; A61B 1/3132; A61B 2017/00287; A61B 2017/3445; A61B 17/3421

(57) ABSTRACT

A surgical device comprises an elongate shaft (12) which extends from a proximal end region to a distal end region thereof, the shaft (12) defining a first lumen (14) for receiving an elongate camera device therein, a second lumen (16) for receiving a tissue manipulation device therein, and a third lumen (18) for receiving a tissue retrieval device therein, the first, second and third lumens (14, 16, 18) extending from the proximal end region to the distal end region of the elongate shaft, and having respective open ends at the distal end region of the elongate shaft. The surgical device further comprises an elongate camera device which extends along the first lumen, a tissue manipulation device which extends along the second lumen, and a tissue retrieval device which extends along the third lumen.

### BACKGROUND OF THE INVENTION

Endoscopic and laparoscopic surgical techniques are well-known, particularly for abdominal operations, and make use of a camera (an endoscope or a laparoscope) inserted into the body of a patient via a relatively small incision, or a natural orifice such as the urethra. Other tools and devices are inserted into the body of the patient also through relatively small incisions. The camera allows the surgeon to view the tissue needing surgery without the need for large incisions required by traditional surgery techniques. Endoscopic surgery, and laparoscopic surgery are sometimes referred to as “keyhole surgery” or “minimally invasive surgery” due to the small size of incisions required.

In procedures that involve removal of at least part of an organ, or a tumour associated with an organ, there is a need for a retrieval bag that can carry the removed tissue from the body cavity. For example, procedures on organs such as the bladder, gall bladder, appendix, colon, spleen, ovary, lymph nodes, lung, etc. may require tissue removal.

The retrieval bag systems currently on the market generally involve totally disposable instruments. For example, U.S. Pat. No. 5,192,284 discloses a surgical collector and extractor for specimen removal through a cannula in a laparoscopic procedure. The device includes a flexible bag for collection purposes. U.S. Pat. No. 5,190,555 discloses a tissue collection device having a flexible bag activated by a drawstring. Other types of tissue retrieval bags are disclosed in, for example, WO2016079752 which is an isolation bag, and WO2016028789 which discloses a bag with two open ends.

Typically, when performing tissue removal using endoscopic or laparoscopic techniques, it is necessary to have three separate devices inserted into the patient; the camera, a grasping/cutting device and a tissue retrieval bag. This means that at least three incisions need to be made in the patient, or an enlarged incision. Multiple incisions and/or enlarged incisions are not desirable for a patient.

Accordingly, it is desirable to provide a new and improved endoscopic/laparoscopic device.

### SUMMARY OF THE INVENTION

Aspects of the present invention are set out in the attached claims.

According to an aspect of the present invention, there is provided a surgical device comprising an elongate shaft which extends from a proximal end region to a distal end region thereof, the shaft defining a first lumen for receiving an elongate camera device therein, a second lumen for receiving a tissue manipulation device therein, and a third lumen for receiving a tissue retrieval device therein, the first, second and third lumens extending from the proximal end region to the distal end region of the elongate shaft, and having respective open ends at the distal end region of the elongate shaft; an elongate camera device which extends along the first lumen; a tissue manipulation device which extends along the second lumen; and a tissue retrieval device which extends along the third lumen.

In one example, the tissue retrieval device comprises a tissue retrieval bag, and an actuation device connected with the tissue retrieval bag and operable to move the tissue retrieval bag between a stowed configuration and a deployed configuration, the tissue retrieval bag being at least partially located within the third lumen of the elongate shaft in the stowed configuration, and being external to the third lumen in the deployed configuration.

In one such example, the actuation device comprises an actuation cord which extends around the end opening of the retrieval bag, and along the third lumen of the shaft through the open end thereof; and an actuation grip located at the proximal end region of the elongate shaft and operable to actuate the actuation cord from a stowed position in which the retrieval bag is at least partially stowed in the lumen of the shaft, and a deployed position in which the retrieval bag extends outside of the lumen of the shaft.

In one example, the third lumen defines a longitudinal axis, and comprises a proximal portion which extends from the proximal end region of the elongate shaft along the longitudinal axis, and a distal portion which extends from the distal end region of the elongate shaft along the longitudinal axis, wherein the proximal portion and the distal portion are contiguous, and wherein the proximal portion has a first dimension in a direction substantially perpendicular to the longitudinal axis of the third lumen, and wherein the distal portion has a second dimension greater than the first dimension in a direction substantially perpendicular to the longitudinal axis of the third lumen.

In one such example, the proximal portion of the third lumen is substantially circular in longitudinal cross-section.

In one such example, the distal portion of the third lumen is substantially oval or lozenge-shaped in longitudinal cross-section.

In one example, one of the first, second and third lumens is arranged to provide a suction pathway for fluids contained in the retrieval bag.

Such a surgical device is suitable for use in endoscopic surgical procedures, wherein the camera device is an endoscope, or is suitable for use in laparoscopic surgical procedures, wherein the camera device is a laparoscope.

Such a device is particularly suitable for use in transurethral bladder tumour removal.

Devices embodying the principles of the present invention may be provided for use in endoscopic or laparoscopic surgical procedures.

### BRIEF DESCRIPTION OF THE DRAWINGS

FIG. 1 is a schematic overview of a tissue retrieval device in a stowed configuration;

FIG. 2 is a schematic overview of a tissue retrieval device in a deployed configuration;

FIG. 3 is a schematic end view of a device shaft embodying one aspect of the present invention;

FIG. 4 is a schematic end view of another device shaft embodying one aspect of the present invention;

FIG. 5 is a schematic partial cross-sectional view of the device shaft of FIGS. 3 and 4; and

FIG. 6 is a schematic partial view of part of the device shaft of FIGS. 3, 4 and 5.

### DETAILED DESCRIPTION OF THE PREFERRED EMBODIMENTS

Endoscopic or laparoscopic surgical techniques that are undertaken to remove tissue from a patient make use of a camera device, a tissue grasping/cutting device and a tissue removal device.

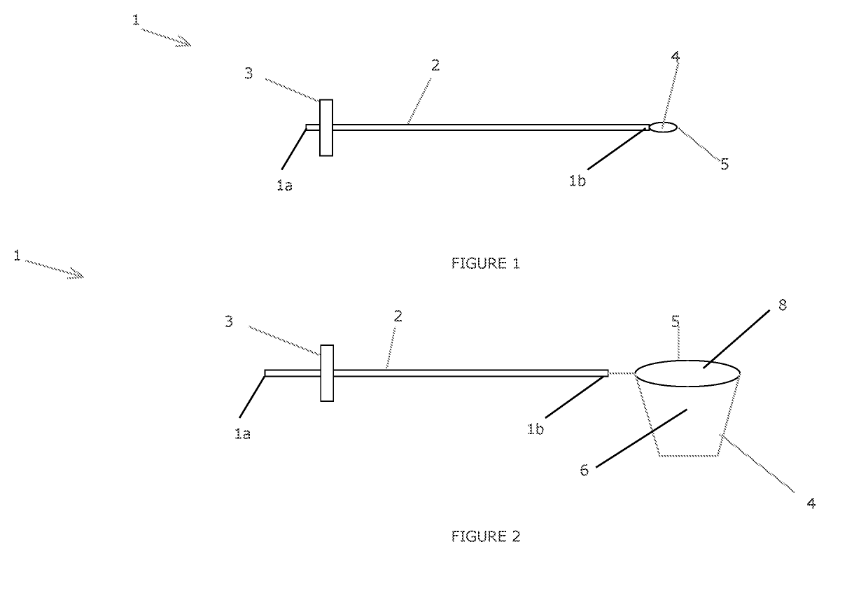

FIG. 1 is a schematic overview of a tissue retrieval device 1 in a stowed configuration. The device 1 comprises an elongate hollow shaft 2 which defines a lumen therealong. The lumen extends from a proximal end 1a of the device 1 to a distal end 1b of the device 1. The lumen has respective open ends at the proximal and distal ends 1a and 1b of the device 1.

An actuation grip 3 (shown schematically for the sake of clarity) is located at the proximal end 1a of the device 1. A tissue retrieval bag 4 is in a stowed position substantially completely within the lumen of the hollow shaft 2 in the stowed configuration shown in FIG. 1. An actuation cord 5 extends around the retrieval bag 4 (to be described in more detail below), and extends through the open end of the lumen, along the lumen to the actuation grip 3. The actuation grip 3 engages with the actuation cord 5. The actuation cord 5 may be any suitable flexible cord, for example may be provided by a wire of suitable stiffness.

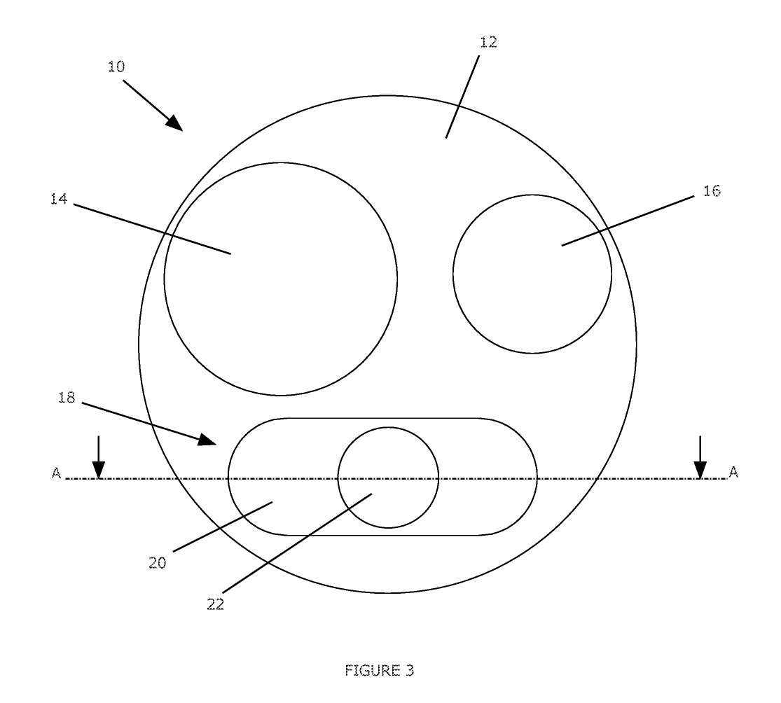

The shaft 2, actuation grip 3 and actuation cord 5 can be referred to as an actuation device for the retrieval bag 4. The actuation grip 3 is movable between a stowed position (as shown in FIG. 1) in which the retrieval bag 4 is substantially contained within the lumen, and a deployed position (as shown in, and to be described with reference to, FIG. 2) in which the retrieval bag 4 is external to the lumen.

FIG. 2 illustrates the tissue retrieval device 1 of FIG. 1 in a deployed configuration. In this deployed configuration, the retrieval bag 4 is external to the lumen, having been moved from the stowed position by movement of the actuation cord 5 along the lumen such that the bag 4 is pushed out of the lumen. In the deployed configuration, the bag 4 expands to provide an inner volume 6. The inner volume 6 has an open aperture 8 through which tissue passes for retrieval. The actuation cord 5 serves to maintain the open aperture 8 in an open position.

Endoscopic and laparoscopic surgical techniques that make use of a camera and a tissue grasper/cutter require multiple incisions to be made in the patient. In accordance with the principles of embodiments of the present invention, a surgical device is provided that has an elongate shaft that is able to provide multiple lumens for the various devices required for a procedure. Such a combined device is particularly suited for use in bladder tumour removal procedures, and for retrieving a tissue sample without seeding of cells from that sample. This is particularly important when the sample is of a tumour.

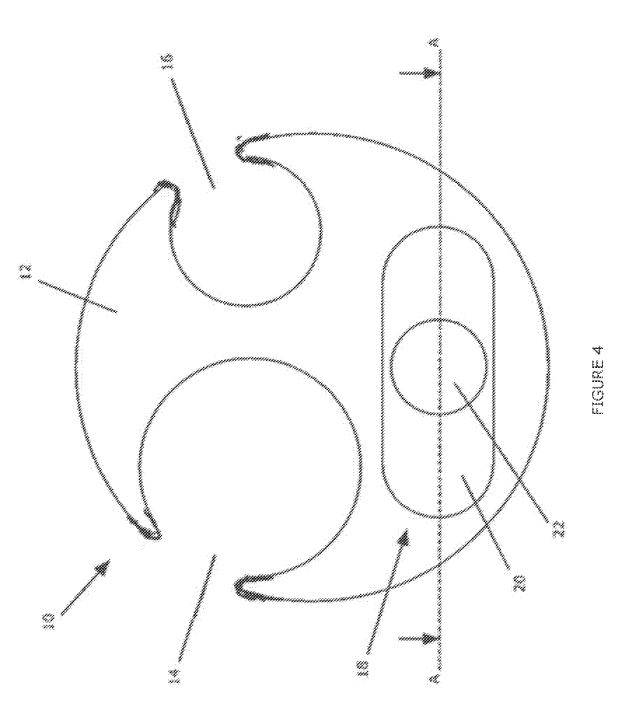

FIG. 3 is a schematic end view of an elongate shaft 12 for a surgical device 10 embodying an aspect of the present invention. The shaft 12 is of extends from a proximal end region to a distal end region. FIG. 3 illustrates an end view of the distal end region. The shaft 12 defines a number of lumens that extend from the proximal end region to the distal end region. In the example shown in FIG. 3, three lumens are provided, each of which provides a location for a device, and each of which has an open end at the distal end region of the surgical device. The devices are not shown in FIG. 3 for the sake of clarity. An alternative embodiment is shown in FIG. 4 in which the first and second lumens have a partially open side portion.

A first lumen 14 is of appropriate size for receiving an elongate camera device, such as an endoscope or a laparoscope. The camera device extends along and through the first lumen 14, so that images can be captured by the camera device from the distal end region of the shaft 12.

A second lumen 16 is of appropriate size for receiving a tissue manipulation device. The tissue manipulation device extends along the second lumen, and out of the distal end region thereof, in order that the surgeon using the device is able to grasp and manipulate tissue adjacent the distal end region of the surgical device. The camera device allows the surgeon to monitor the action of the manipulation device.

A third lumen 18 extends along the elongate shaft 12 for receiving a tissue retrieval device therein. The tissue removal device can be any appropriate device, for example a device as illustrated in FIGS. 1 and 2. The third lumen 18 comprises first and second portions 20 and 22. The first portion 20 extends from the proximal end of the elongate shaft 12 towards the distal end, and the second portion 22 extends from the distal end of the first portion 20 to the distal end of the elongate shaft 12. The first and second portions 20 and 22 of the third lumen 18 are contiguous and form a continuous lumen from the proximal end region to the distal end of the elongate shaft 12. The second portion 22 provides an open distal end of the third lumen 18 at the distal end of the elongate shaft 12.

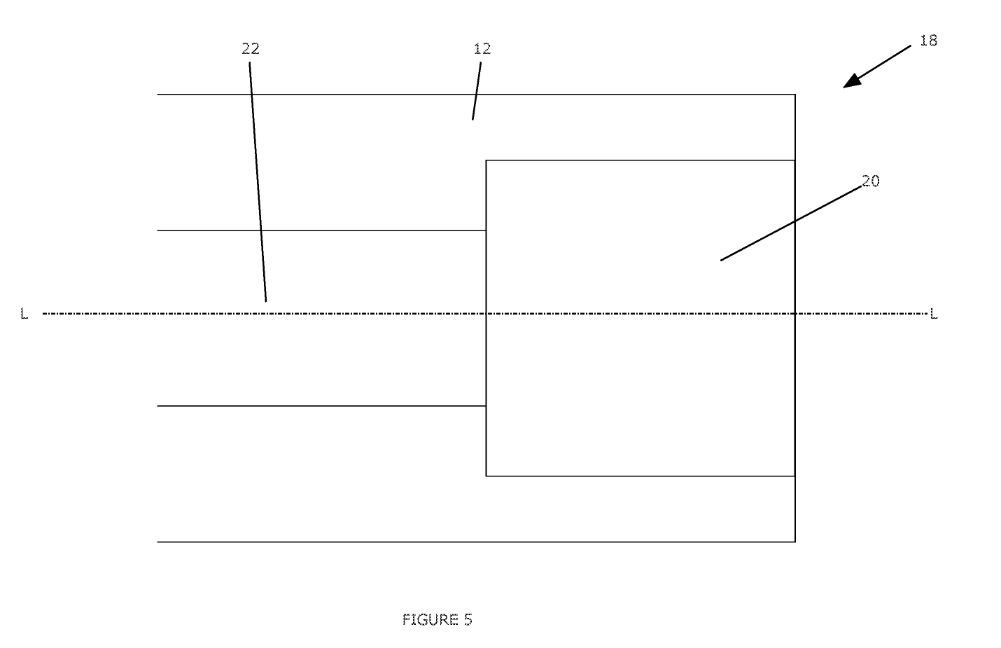

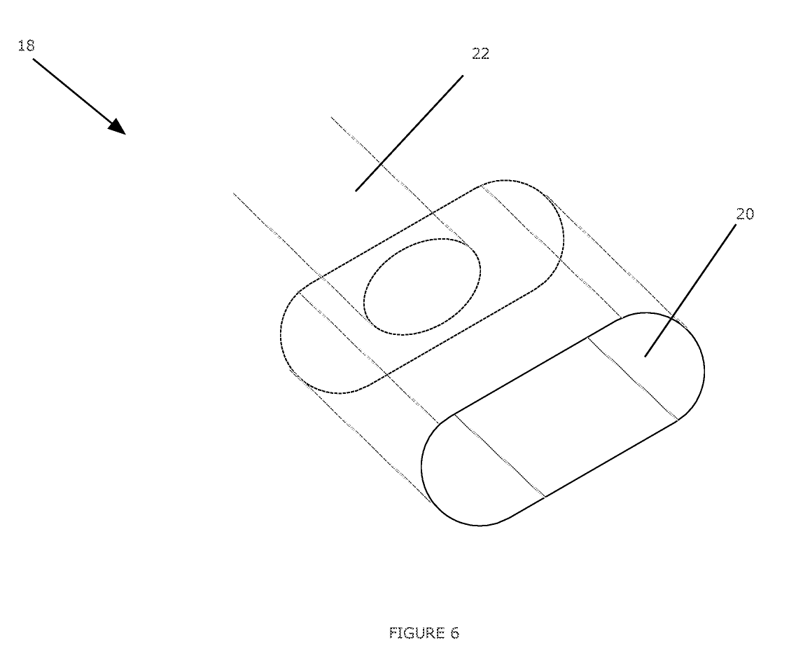

FIG. 5 illustrates a partial transverse cross-sectional view of the distal end region of the elongate shaft 12, taken along line A-A of FIG. 3 or 4, and FIG. 6 illustrates a perspective view of the distal end region of the third lumen 18. As illustrated in FIGS. 5 and 6, the second portion 22 of the third lumen 18 the first and second portions 20 and 22 have respective first and second dimensions in a direction substantially perpendicular to the longitudinal axis L-L of the third lumen 18. The second dimension (of the second portion 22) is greater than the first dimension (of the first portion 20). The second portion 22 has a greater longitudinal cross-sectional area than the first portion 20. In one example, the second portion 22 is oval or lozenge-shaped in longitudinal cross-section. In one example, the first portion 20 is circular in longitudinal cross-section.

As such, the second portion 22 provides a location in which the retrieval bag of the tissue retrieval device can be stowed. The retrieval bag is fully or partially stowed within the second portion 22 by moving the actuation cord of the retrieval device into the stowed position, thereby pulling the retrieval bag partially or fully into the second portion 22. The change in cross sectional area between the second and first portions 22 and 20 provides the third lumen 18 with an effective stopper for the retrieval bag, which prevents the bag being pulled further than the required distance within the third lumen 18.

The third lumen 18 may also provide a suction pathway for the efficient removal of fluids and/or other material through the elongate shaft 12. In other examples. The first or second lumen may be used for providing such a suction pathway,

One embodiment of the present invention provides a multi-lumen elongate shaft that fits within the diameter of existing resectoscope outer sheaths, thereby enabling more efficient use of the space within the existing sheaths. Such space efficiency enables the provision of multiple tools and devices within a single shaft.

1. A surgical device comprising:
an elongate shaft which extends from a proximal end region to a distal end region thereof, the shaft defining a first lumen for receiving an elongate camera device therein, a second lumen for receiving a tissue manipulation device therein, and a third lumen for receiving a tissue retrieval device therein, the first, second and third lumens extending from the proximal end region to the distal end region of the elongate shaft, and having respective open ends at the distal end region of the elongate shaft;
an elongate camera device which extends along the first lumen;
a tissue manipulation device which extends along the second lumen; and
a tissue retrieval device which extends along the third lumen.

2. A surgical device as claimed in claim 1, wherein tissue retrieval device comprises a tissue retrieval bag, and an actuation device connected with the tissue retrieval bag and operable to move the tissue retrieval bag between a stowed configuration and a deployed configuration, the tissue retrieval bag being at least partially located within the third lumen of the elongate shaft in the stowed configuration, and being external to the third lumen in the deployed configuration.

3. A surgical device as claimed in claim 2, wherein the actuation device comprises:
an actuation cord which extends around the end opening of the retrieval bag, and along the third lumen of the shaft through the open end thereof; and
an actuation grip located at the proximal end region of the elongate shaft and operable to actuate the actuation cord from a stowed position in which the retrieval bag is at least partially stowed in the lumen of the shaft, and a deployed position in which the retrieval bag extends outside of the lumen of the shaft.

4. A surgical device as claimed in claim 1, wherein the third lumen defines a longitudinal axis, and comprises a proximal portion which extends from the proximal end region of the elongate shaft along the longitudinal axis, and a distal portion which extends from the distal end region of the elongate shaft along the longitudinal axis, wherein the proximal portion and the distal portion are contiguous, and wherein the proximal portion has a first dimension in a direction substantially perpendicular to the longitudinal axis of the third lumen, and wherein the distal portion has a second dimension greater than the first dimension in a direction substantially perpendicular to the longitudinal axis of the third lumen.

5. A surgical device as claimed in claim 4, wherein the proximal portion of the third lumen is substantially circular in longitudinal cross-section.

6. A surgical device as claimed in claim 4, wherein the distal portion of the third lumen is substantially oval or lozenge-shaped in longitudinal cross-section.

7. A surgical device as claimed in claim 1, wherein one of the first, second and third lumens is arranged to provide a suction pathway for fluids contained in the retrieval bag.

8. A surgical device as claimed in claim 1, for use in endoscopic surgical procedures, wherein the camera device is an endoscope.

9. A surgical device as claimed in claim 1, for use in laparoscopic surgical procedures, wherein the camera device is a laparoscope.

10. A surgical device as claimed in claim 1, for use in bladder tumour removal.

11. An elongate shaft for a surgical device as claimed in claim 1, wherein the elongate shaft extends from a proximal end region to a distal end region thereof, and defines:
a first lumen for receiving an elongate camera device therein;
a second lumen for receiving a tissue manipulation device therein; and
a third lumen for receiving a tissue retrieval device therein,
wherein the first, second and third lumens extend from the proximal end region to the distal end region of the elongate shaft, and have respective open ends at the distal end region of the elongate shaft.
